(19) United States
(12) Patent Application Publication
(10) Pub. No.: US 2018/0325784 A1
(43) Pub. Date: Nov. 15, 2018

(54) Self Assembling Skin Care Emulsions

(71) Applicant: Lubrizol Advanced Materials, Inc., Cleveland, OH (US)

(72) Inventors: Krishnan Chari, Hudson, OH (US); Brian Figura, Cleveland, OH (US); Anchuu Wu, Broadview Heights, OH (US); Robert Jacobs, Olmsted Falls, OH (US)

(21) Appl. No.: 15/775,578

(22) PCT Filed: Nov. 16, 2016
(86) PCT No.: PCT/US2016/062278

§ 371 Date: May 11, 2018

Related U.S. Application Data
(60) Provisional application No. 62/257,501, filed on Nov. 19, 2015.

(51) Int. Cl. A61K 8/06; A61K 8/25; A61K 8/29; A61K 8/27; A61K 8/85; A61K 8/81; A61Q 19/00

(52) U.S. Cl. CPC A61K 8/06; A61K 8/25; A61K 8/29; A61K 2800/621; A61K 8/85; A61K 8/8129; A61Q 19/00; A61K 8/27

(57) ABSTRACT

The disclosed technology relates to near monodisperse and tunable emulsion droplets that can be uniformly coated onto hydrophilic and hydrophobic surfaces and provide superior protection from ultra-violet radiation as well as water resistance and improved actives delivery.

### BACKGROUND OF THE INVENTION

The disclosed technology relates to near monodisperse and tunable emulsion droplets that can be uniformly coated onto both hydrophilic surfaces and hydrophobic surfaces, such as skin, and provide superior protection from ultra-violet radiation as well as water resistance and improved actives delivery.

Emulsions are ubiquitous in skin care applications: they are relied on to deliver sunscreen actives, pigments, moisturizing oils, and lipophilic actives. However, selecting the proper emulsifier for a given application can be challenging, as one must consider the hydrophilic-lipophilic balance of the oil phases in question. Once created, many common formulation processes result in an emulsion with a poly-disperse size distribution; or in other words, an emulsion having droplets of widely varying size distribution. Additionally, depending upon factors such as the choice of emulsifier, stabilizer, and oil solubility in the continuous phase, the size distribution may change as a function of time due to droplet coalescence or Ostwald ripening.

Conventional emulsifiers are used in emulsions usually at around 1 wt %. The process for using conventional emulsifiers in oil-in-water emulsions is to dissolve the emulsifiers in the oil phase with heat and mixing, and then add the oil phase to the water phase with mixing. Such a process results in an emulsion with a poly-disperse particle size distribution. The use of poly-disperse droplet size distribution is undesirable in skin-care applications as it is less likely to lead to uniform coverage of the skin by the oil phase. An example of poly-disperse Pickering emulsions can be found, for example, in U.S. Pat. No. 6,703,032 to Gers-Barlag et al., issued Mar. 9, 2004, which Pickering emulsions contain only inorganic particle stabilizers. Similarly, EP 1,958,687 B1, issued 23 Nov. 2011, teaches poly-disperse Pickering emulsions with ionic polymers and organic particles.

In contrast, it would be desirable to start with an emulsion whose particle size distribution (PSD) was monodisperse; that is, an emulsion having droplets of consistent size with little variation in size from particle to particle. Such a distribution should lead to a more uniform (and hence efficacious) deposition of oil (along with whatever actives may be dissolved in the oil phase).

### SUMMARY OF THE INVENTION

The disclosed technology, therefore, solves the problem of obtaining a uniform film of an oil on a substrate by providing a Pickering emulsion having a uniform dispersion of a stabilizer system.

In one aspect, the disclosed technology provides a new type of Pickering emulsion. The Pickering emulsion can be a finely dispersed water-in-oil or oil-in-water system. The Pickering emulsion can include a cosmetically or pharmaceutically acceptable organic phase; an aqueous phase; and a uniform dispersion of a stabilizer system.

In an embodiment, the Pickering emulsion taught herein can generate a uniform film as evidenced by the generation of a diffraction pattern exhibiting one or more distinct rings rather than diffuse scattering, for example as seen in FIG. 1, when coated as a thin film on a transparent surface and illuminated with a coherent beam of light, such as a laser. In some embodiments, the transparent surface can be a hydrophobic surface.

In another embodiment, the stabilizer system of the Pickering emulsion can include a non-ionic polymer and inorganic solid particles. In one embodiment, the non-ionic polymer stabilizer can include a polyester polymer, such as the reaction product of a diacid and a diol. For example, the polymer may be a polypropylene glycol adipate, such as dipropylene glycol adipate. The polymer can also be a polypropylene glycol glutarate, such as dipropylene glycol glutarate. In another embodiment, the non-ionic polymer can include an adipic acid co-methylaminoethanol (“MAE”) copolymer. In an embodiment, the inorganic solid particles can include zinc, silica, titanium dioxide, or combinations thereof.

Also included as an embodiment is a Pickering emulsion prepared by the steps comprising: A) dissolving the stabilizer system into at least one of the aqueous phase or the cosmetically or pharmaceutically acceptable organic phase; B) slowly adding into the solution from step A) the other of the aqueous phase or the cosmetically or pharmaceutically acceptable organic phase with mixing; and C) subjecting the mixture of step B) to shear sufficient to induce limited coalescence.

In an embodiment, there is provided a cosmetically or pharmaceutically acceptable skin-care composition containing the Pickering emulsion as described herein, in a continuous phase comprising water, or a cosmetically or pharmaceutically acceptable organic phase. In a further embodiment, the cosmetically or pharmaceutically acceptable skin-care composition can include at least one cosmetically or pharmaceutically acceptable additive, such as, for example, a UV absorber.

The cosmetically or pharmaceutically acceptable skin-care composition can result in a reduction in transmission of UV light to a substrate coated with the composition.

Another aspect of the technology includes a process for producing a Pickering emulsion having a uniform size distribution. The process can include A) dissolving or dispersing a stabilizer system into an aqueous phase; B) mixing the product of step A) with a cosmetically or pharmaceutically acceptable organic phase; and C) subjecting the mixture of step B) to shear sufficient to induce limited coalescence.

### BRIEF DESCRIPTION OF THE FIGURES

FIG. 1 represents a diffraction pattern caused by Fraunhofer diffraction of light indicating a disordered coating of droplets as would be expected in prior art conventional or Pickering emulsions for personal and home care applications.

FIG. 2 represents a diffraction pattern caused by Fraunhofer diffraction of light indicating a close-packed ordered monolayer of the Pickering emulsion as disclosed herein.

### DETAILED DESCRIPTION OF THE INVENTION

Various preferred features and embodiments will be described below by way of non-limiting illustration.

The technology at hand is directed in part to a new type of Pickering emulsion, methods of preparing the Pickering emulsion, and uses of the Pickering emulsion in personal and home care applications.

The Pickering emulsions provided herein can be either finely dispersed water-in-oil systems, or finely dispersed oil-in-water systems. In either event, the Pickering emulsion will contain both a cosmetically or pharmaceutically acceptable organic phase (sometimes referred to herein as an “oil”) and an aqueous phase, as well as a uniform dispersion of a stabilizer system.

The organic phase of the Pickering emulsion is generally a hydrocarbon, such as an oil, but may include emollients, fragrances and the like. In essence, the organic phase is any organic material that may be employed in a cosmetic or pharmaceutical emulsion.

Non-limiting examples of an organic phase include mineral oils; petrolatums; vegetable oils (including nut oils); hydrogenated vegetable oils; essential oils; algae oils; fish oils; fatty alcohols; fatty acids; fatty acid and fatty alcohol esters; alkoxylated fatty alcohols; alkoxylated fatty acid esters; benzoate esters; Guerbet esters; alkyl ether derivatives of polyethylene glycols, such as, for example methoxypolyethylene glycol (MPEG); and polyalkylene glycols; lanolin and lanolin derivatives; waxes; and the like, as well as mixtures thereof. The organic phase can be utilized in an amount of from about 10 to about 50 wt. %, or from about 15 to about 30, 0r 40 wt/%.

Mineral oils and petrolatums include cosmetic, USP and NF grades and are commercially available from Penreco under the Drakeol™ and Penreco™ trade names.

Exemplary vegetable oils suitable as an organic phase can include but are not limited to peanut oil, sesame oil, avocado oil, coconut oil, cocoa butter, almond oil, safflower oil, corn oil, cotton seed oil, castor oil, olive oil, jojoba oil, palm oil, palm kernel oil, soybean oil, wheat germ oil, linseed oil, sunflower seed oil; and the mono-, di-, and triglycerides thereof, and hydrogenated derivatives thereof; and mixtures thereof. Exemplary mono-, di- and triglycerides are, for example, caprylic triglyceride, capric triglyceride, caprylic/capric triglyceride, and caprylic/capric/lauric triglyceride, caprylic/capric/stearic triglyceride, and caprylic/capric/linoleic triglyceride.

Ethoxylated mono- and diglycerides of the foregoing vegetable oils are also contemplated, such as, for example, PEG-8 Caprylic/Capric Glycerides.

Essential oils can be employed as an organic phase and can encompass oils having an aromatic essence. Essential oils include, but are not limited to peppermint oil, cedar oil, castor oil, clove oil, geranium oil, lemongrass oil, linseed oil, mint oil, thyme oil, rosemary oil, cornmint oil (Mentha arvensis), garlic oil, anise oil, basil oil, camphor oil, citronella oil, eucalyptus oil, fennel oil, ginger oil, grapefruit oil, lemon oil, lime oil, mandarin oil, orange oil, pine needle oil, pepper oil, rose oil, tangerine oil, tea tree oil, tea seed oil, mineral oil and fish oil.

Suitable fatty alcohol an organic phase include but are not limited to fatty alcohols containing 8 to 50 carbon atoms. Exemplary fatty alcohols include capryl alcohol, pelargonic alcohol, capric alcohol, lauryl alcohol, myristyl alcohol, cetyl alcohol, isocetyl alcohol, stearyl alcohol, isostearyl alcohol, cetearyl alcohol, oleyl alcohol, ricinoleyl alcohol, arachidyl alcohol, icocenyl alcohol, behenyl alcohol, and mixtures thereof.

Suitable fatty acids as the organic phase include but are not limited to fatty acids containing 10 to 50 carbon atoms. Exemplary fatty acids are selected from capric acid, lauric acid, myristic acid, palmitic acid, stearic acid, oleic acid, linoleic acid, arachidic acid, behenic acid, and mixtures thereof.

Suitable fatty acid and fatty alcohol ester organic phases include but are not limited to hexyl laurate, decyl oleate, isopropyl stearate, isopropyl isostearate, butyl stearate, octyl stearate, ethylhexyl stearate, cetyl stearate, myristyl myristate, octyldodecyl stearoylstearate, octylhydroxystearate, diisopropyl adipate, isopropyl myristate, isopropyl palmitate, ethyl hexyl palmitate, isodecyl oleate, isodecyl neopentanoate, diisopropyl sebacate, isostearyl lactate, isostearyl hydroxy stearate, diisostearyl fumarate, lauryl lactate, diethyl hexyl maleate, PPG-14 butyl ether and PPG-2 myristyl ether propionate, ethylhexyl octanoate, cetearyl octanoate, cetearyl ethylhexanoate, and mixtures thereof.

Alkoxylated fatty alcohols are ethers formed from the reaction of a fatty alcohol with an alkylene oxide, generally ethylene oxide or propylene oxide. Suitable ethoxylated fatty alcohols are adducts of fatty alcohols and polyethylene oxide. In one aspect the ethoxylated fatty alcohols can be represented by the formula R—(OCH₂CH₂)ₙ—OH wherein R represents the linear or branched aliphatic residue of the parent fatty alcohol and n represents the number of molecules of ethylene oxide. In another aspect, R is derived from a fatty alcohol containing 8 to 40 carbon atoms. In one aspect n is an integer ranging from 2 to 100, 3 to 80 in another aspect, and 3 to 50 in a further aspect. In a still further aspect, R is derived from a fatty alcohol organic phase set forth above. Exemplary ethoxylated fatty alcohols can include but are not limited to capryl alcohol ethoxylate, lauryl alcohol ethoxylate, myristyl alcohol ethoxylate, cetyl alcohol ethoxylate, stearyl alcohol ethoxylate, cetearyl alcohol ethoxylate oleyl alcohol ethoxylate, and, behenyl alcohol ethoxylate, wherein the number of ethylene oxide units in each of the foregoing ethoxylates can range from 2 and above in one aspect, and from 2 to about 150 in another aspect. It is to be recognized that the propoxylated adducts of the foregoing fatty alcohols and ethoxylated/propoxylated adducts of the foregoing fatty alcohols are also contemplated. More specific examples of alkoxylated alcohols are beheneth 5-30 (the 5-30 meaning the number of repeating ethylene oxide or propylene oxide units), Ceteareth 2-100, Ceteth 1-45, Cetoleth 24-25, Choleth 10-24, Coceth 3-10, C9-11 Pareth 3-8, C11-15 pareth 5-40, C11-21 Pareth 3-10, C12-13 Pareth 3-15, Deceth 4-6, Dodoxynol 5-12, Glycereth 7-26, Isoceteth 10-30, Isodeceth 4-6, Isolaureth 3-6, Isosteareth 3-50, Laneth 5-75, Laureth 1-40, Nonoxynol 1-120, Nonoxynol 5-150, Octoxynol 3-70, Oleth 2-50, Steareth 2-100, Trideceth 2-10, and so on.

Alkoxylated fatty acids are formed when a fatty acid is reacted with an alkylene oxide or with a pre-formed polymeric ether. The resulting product may be a monoester, diester, or mixture thereof. Suitable ethoxylated fatty acid ester organic phases suitable for use are products of the addition of ethylene oxide to fatty acids. The product is a polyethylene oxide ester of a fatty acid. In one aspect, the ethoxylated fatty acid esters can be represented by the formula R—C(O)O(CH₂CH₂O)ₙ—H, wherein R represents the linear or branched aliphatic residue of a fatty acid and n represents the number of molecules of ethylene oxide. In another aspect, n is an integer ranging from 2 to 50, 3 to 25 in another aspect, and 3 to 10 in a further aspect. In still another aspect, R is derived from a fatty acid containing 8 to 30 carbon atoms. In a still further aspect, R and the C(O)O— group is derived from a fatty acid organic phase material set forth above. It is to be recognized that propoxylated and ethoxylated/propoxylated products of the foregoing fatty acids are also contemplated. Exemplary alkoxylated fatty acid esters include but are not limited to capric acid ethoxylate, lauric acid ethoxylate, myristic acid ethoxylate, stearic acid ethoxylate, oleic acid ethoxylate, coconut fatty acid ethoxylate, and polyethylene glycol 400 propoxylated monolaurate, wherein the number of ethylene oxide units in each of the foregoing ethoxylates can range from 2 and above in one aspect, and from 2 to about 50 in another aspect. More specific examples of ethoxylated fatty acids are PEG-8 distearate (the 8 meaning the number of repeating ethylene oxide units), PEG-8 behenate, PEG-8 caprate, PEG-8 caprylate. PEG-8 caprylate/caprate, PEG cocoates (PEG without a number designation meaning that the number of ethylene oxide units ranges from 2 to 50), PEG-15 dicocoate, PEG-2 diisononanoate, PEG-8 diisostearate, PEG-dilaurates, PEG-dioleates PEG-distearates, PEG Ditallates, PEG-isostearates, PEG-jojoba acids, PEG-laurates, PEG-linolenates, PEG-myristates, PEG-oleates, PEG-palmitates, PEG-ricinoleates, PEG-stearates, PEG-tallates, and the like.

Benzoate ester organic phases are selected from but not limited to C₁₂ to C₁₅ alkyl benzoate, isostearyl benzoate, octyl dodecyl benzoate, stearyl benzoate, dipropylene glycol dibenzoate, methyl gluceth-20 benzoate, castor oil benzoate, cetyl ricinoleate benzoate, ethylhexyl hydroxystearate benzoate, dimethicone PEG/PPG20/23 benzoate, and dimethicone PEG-8 benzoate.

Guerbet ester organic phase materials are formed from the esterification reaction of a Guerbet alcohol with a carboxylic acid. Guerbet ester organic phase materials are commercially available from Noveon, Inc. as G-20, G-36, G-38, and G-66.

Lanolin and lanolin derivatives are selected from lanolin, lanolin wax, lanolin oil, lanolin alcohols, lanolin fatty acids, alkoxylated lanolin, isopropyl lanolate, acetylated lanolin alcohols, and combinations thereof. Lanolin and lanolin derivatives are commercially available from Noveon, Inc. under the following trade names Lanolin LP 108 USP, Lanolin USP AAA, Acetulan™, Ceralan™, Lanocerin™, Lanogel™ (product designations 21 and 41), Lanogene™, Modulan™, Ohlan™, Solulan™ (product designations 16, 75, L-575, 98, and C-24), Vilvanolin™ (product designations C, CAB, L-101, and P).

Waxes include those derived from plant, animal/insect, mineral, petroleum and synthetic sources. Synthetically modified natural (plant and animal/insect) waxes are also contemplated. Exemplary plant derived waxes include but are not limited to bayberry wax, candelilla wax, hydrolyzed candelilla wax, carnauba wax, ethoxylated carnauba wax (e.g., PEG-12 carnauba wax), hydrolyzed carnauba wax, carnauba acid wax, hydrogenated castor wax, esparto wax, hydrogenated Japan wax, hydrogenated jojoba oil, jojoba oil esters, sulfurized jojoba oil, ouricury wax, palm kernel wax, and hydrogenated rice bran wax. Exemplary animal/insect derived waxes include but are not limited to beeswax, oxidized beeswax, ethoxylated beeswax (e.g., PEG-6 beeswax, PEG-8 beeswax, PEG-12 beeswax, PEG-20 beeswax), dimethicone copolyol beeswax esters and dimethiconol beeswax ester (e.g. Bis-Hydroxyethoxypropyl Dimethicone Beeswax Esters, Dimethicone PEG-8 Beeswax, and Dimethiconol Beeswax available from Noveon, Inc. under the Ultrabee™ trademark), Chinese wax, shellac wax, spermaceti wax, mink wax, and lanolin wax. Exemplary mineral waxes include but are not limited to ceresin waxes, montan wax, montan acid wax, and ozocerite. Exemplary petroleum waxes include paraffin waxes, such as isododecane and isohexadecane, microcrystalline waxes, and oxidized microcrystalline waxes. Exemplary synthetic waxes include synthetic beeswax, synthetic candelilla wax, synthetic carnauba wax, synthetic Japan wax, synthetic jojoba oil, polyolefin waxes (e.g., polyethylene wax), ethylene glycol diesters or triesters of fatty acids containing 18 to 40 carbon atoms. Mixtures of two or more of the forgoing waxes and classes of waxes are also contemplated.

In some embodiments, the organic phase material can be an emollient such as dioctyl/dicapryl ether.

In some embodiments, the organic phase material can be an organic sunscreen.

The organic phase material can also be a fragrance, whether naturally derived or synthetically derived.

In some embodiments, the oil comprises, consists essentially of, or consists of a mineral oil. In other embodiments, the oil comprises, consists essentially of, or consists of a vegetable oil.

In some embodiments, the organic phase material can be any of the common oils employed in cosmetic formulations, such as, for example, castor oil, coco-glycerides (di, tri), caprylic/capric triglyceride, coconut oil, sweet almond oil, sunflower oil, isopropyl palmitate, cetearyl ethylhexanoate, ethylhexyl stearate, jojoba oil, isododecane, mineral oil, isohexadecane, dioctyl/dicapryl ether, or mixtures thereof.

It will be recognized by those of skill in the art that the various organic phase materials mentioned above may be considered in various categories. Thus, the exemplary descriptions of the various organic phase materials is not meant as a single definition of any one specific organic phase material. There are many more organic phase materials not referenced herein, but nonetheless would be expected to be suitable as an organic phase hereunder when employing the formulating principles set forth herein. In addition, the organic phase materials may be used alone or in combination with other organic phase materials.

### Stabilizer System

The Pickering emulsion described herein will include a uniform dispersion of a stabilizer system; that is, a stabilizer system uniformly dispersed in the aqueous or organic phase, as the case may be.

A “uniform dispersion,” as used herein, refers to a dispersion in which dispersed droplets or particles (referred to collectively as particles) in the dispersion have a size distribution that is uniform, or in other words, a distribution in which the particles are all of consistent size with little variation from particle to particle. In an embodiment, the uniform dispersion can have a particle size distribution having, for example, a coefficient of variation (cv), defined as the standard deviation of the distribution divided by the arithmetic mean, of less than about 0.25, preferably less than 0.2 and most preferably less than 0.15.

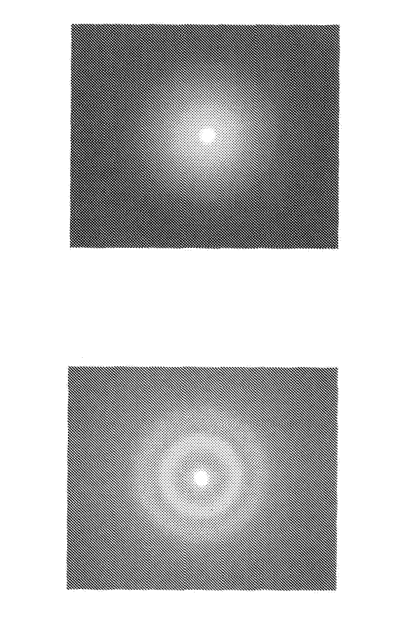

In some embodiments it may be difficult to measure the particle sizes of the uniform dispersion. Thus, in another embodiment, “uniform dispersion” can refer to a dispersion having a particle size distribution that is uniform as evidenced by the generation of a diffraction pattern exhibiting one or more distinct rings, for example, as illustrated in FIG. 2, rather than individual bright points, for example, as illustrated in FIG. 1, when coated as a thin film on a transparent surface and illuminated with a coherent beam of light, such as a laser. Such a diffraction pattern may be referred to as Fraunhofer diffraction. The phenomenon of Fraunhofer diffraction is described more fully by Lisensky et al. Journal of Chemical Education, vol. 68, February 1991. By “thin film” it is meant a film having, for example, a thickness of about 50 microns or less, or 25 microns or less, or in some cases 10 microns or less, and in an embodiment “thin film” refers to a film having the thickness of a monolayer of the Pickering emulsion droplets. The thin film can be, for example, coated onto a hydrophobic surface, such as, for example, a surface molded from polymethyl methacrylate (“PMMA”), such as Helioplates™ manufactured by HelioScreen, the surface of the skin, surface, or a hydrophilic surface, such as, for example, surface treated glass. In another embodiment, the thin film can be coated, for example, onto a hydrophilic surface, such as standard laboratory glass slides.

In an embodiment, the stabilizer system can include a two-part stabilizer of a polymer, such as a non-ionic polymer, and inorganic solid particles, such as, for example, zinc oxide, titanium dioxide, or silica. In some embodiments, the inorganic solid particle can be coated with silica. Silica coated titanium dioxide and zinc oxide are commercially available.

The polymer of such a two-part stabilizer system can include, for example, non-ionic polymers. Examples include both naturally occurring substances such as proteins, protein derivatives, cellulose derivatives (for example cellulose esters), gelatins and gelatin derivatives, polysaccharides, casein, and the like, and synthetic water permeable colloids such as poly(vinyl lactams), polyesters, acrylamide polymers, latex, poly(vinyl alcohol) and its derivatives, hydrolyzed polyvinyl acetates, polymers of alkyl and sulfoalkyl acrylates and methacrylates, polyamides, polyvinyl pyridine, acrylic acid polymers, maleic anhydride copolymers, polyalkylene oxide, methacrylamide copolymers, polyvinyl oxazolidinones, maleic acid copolymers, vinyl amine copolymers, methacrylic acid copolymers, acryloyloxyalkyl acrylate and methacrylates, vinyl imidazole copolymers, and vinyl sulfide copolymers.

The polymer in the stabilizer system should be a cosmetically or pharmaceutically acceptable polymer. In other words, the polymer should be toxicologically acceptable for use on humans. In an embodiment, the polymer can be purified copolymer of adipic acid co-methylaminoethanol (“MAE”) that is suitable for cosmetic or pharmaceutical use. However, it is well known that secondary amines can form nitrosamine, which are toxicologically harmful (known to be carcinogenic). As such, unless well purified, amine polymers are not preferred polymers in the stabilizer system.

Other polymers that can be utilized include dextran, gum arabic, zein, casein, pectin, collagen derivatives, collodion, agar-agar, arrowroot, albumin, and the like. Still other useful polymers are water soluble polyvinyl compounds such as polyvinyl alcohol, polyacrylamide, poly(vinylpyrrolidone), and the like.

Other organic binders such as polyvinyl alcohol (PVA) or polyethylene oxide (PEO) can be used as components of the dispersion.

Polyesters suitable as the non-ionic polymer can include, for example, the reaction product of diacids and diols. Suitable diacids for preparing the polyesters can include, for example, ethanedioic acid (oxalic acid), propanedioic acid (malonic acid), butanedioic acid (succinic acid), pentanedioic acid (glutaric acid), hexanedioic acid (adipic acid), heptanedioic acid (pimelic acid), octanedioic acid (suberic acid), nonanedioic acid (azelaic acid), decanedioic acid (sebacic acid), un-decanedioic acid, dodecanedioic acid, hexadecanedioic acid, the mono-unsaturated diacids, such as maleic acid, fumaric acid, glutaconic acid, trau-matic acid, di-unsaturated muconic acid, glutinic acid, the branched citracon-ic acid, mesaconic acid, itaconic acid, tartronic acid, tartaric acid, arabinaric acid, saccharic acid, mesoxalic acid, oxaloacetic acid and acetonedicarboxylic acid. The diacids can be in the form of free diacid or diacid anhydrides, both of which are encompassed by the term “diacid.” Suitable diols for preparing the polyesters can include, for example ethanediol, polyethylene glycol, propanediol, polypropylene glycol, butanediol, polybutylene glycol, polytetrahydrofuran, pentanediol, hexanediol, polyglycol copolymers, glycerol, polyglycerol, and glycol glycerine copolymers, trimethylolpropane, pentaerythritol, and other polyols or carbohydrates, such as fructose, glucose, sucrose and their isomers and derivatives. In an embodiment, the polyester can include the reaction product of polypropylene glycol and adipic acid.

In an embodiment, the polyester can have a structure of formula [O(CO)R(CO)OR′]ₙ, where O(CO)R(CO)O is derived from a diacid and R is an aliphatic or aromatic containing hydrocarbyl group of from about 1 to 10 carbon atoms, or 1 to 5 carbon atoms, or 1, 2 or 3 carbon atoms, and R′ is derived from a diol and is an aliphatic or aromatic containing hydrocarbyl group of from about 1 to 10 carbon atoms, or 1 to 5 carbon atoms, or 1, 2 or 3 carbon atoms, and n is an integer of from about 1 to 20, or 1 to 10, or 1 to 5, or 1, 2 or 3. R may optionally be branched and/or substituted with oxygen or hydroxyl groups, such as in, for example, arabinaric acid, oxaloacetic acid and acetonedicarboxylic acid.

Example polyesters can include, but are not limited to, for example, polyethylene glycol succinate, polyethylene glycol adipate, polyethylene glycol sebacate, polypropylene glycol succinate, polypropylene glycol adipate, polypropylene glycol sebacate, polypropylene glycol glutarate, PEG-PPG succinate, PEG-PPG adipate, PEG-PPG sebacate, hexylene glycol succinate, hexylene glycol adipate, hexylene glycol sebacate, 2-methyl-2,4-pentanediol succinate, 2-methyl-2,4-pentanediol adipate, 2-methyl-2,4-pentanediol sebacate; and the like, and combinations thereof.

The amount of polymer and solid particle may vary depending on which polymer and particle is employed. It has been found that adjusting the level of polymer and particle in the stabilizer system can control the final emulsion particle size distribution. The higher the load of stabilizer system, the smaller the particle size. In general, each of the polymer and particle may be present individually at from about 1 to about 10 wt %, or from about 1 to about 5 wt %, generally with a ratio of from about 1:5 to about 5:1 polymer to particle, or even a ratio of from about 1:4 to about 4:1, or about 1:3 to about 3:1, and even from about 1:2 to about 2:1 or about 1:1 to about 2:1 polymer to particle.

### Process

In one embodiment of the technology there includes a process for producing a Pickering emulsion having a stabilizer system of uniform size distribution. The process includes inducing limited coalescence of the emulsion.

The limited coalescence technique is used and described by Thomas H. Whitesides and David S. Ross in “J. Colloid Interface Science” 169.48-59 (1995). The limited coalescence method can include a “suspension polymerization” technique and a “polymer suspension” technique. The suspension method includes adding polyaddition polymerizable monomer or monomers to an aqueous medium containing a particulate suspending agent to form a discontinuous (oil droplet) phase in a continuous (aqueous) phase. The mixture is subjected to shearing forces, by agitation, homogenization and the like to reduce the size of the droplets. After shearing is stopped, an equilibrium is reached with respect to the size of the droplets as a result of the stabilizing action of the particulate suspending agent in coating the surface of the droplets. In this process, polymerization is completed to form an aqueous suspension of polymer particles. This process is described in U.S. Pat. Nos. 2,932,629; 5,279,934; and 5,378,577; which are incorporated herein by reference.

The suspension polymerization process, as its name implies, is employed to produce polymer and therefore requires polymerization. While polymerization may be useful to prepare the polymer of the stabilizer system in situ, it has surprisingly been found that the technique involving the addition of shear forces to reduce particles sizes works well for the instant technology to achieve limited coalescence of the stabilizer system in the Pickering emulsion. Thus, in an embodiment, the process for producing the Pickering emulsion can include the steps of A) dissolving or dispersing a stabilizer system into an aqueous phase; B) mixing the aqueous phase of step A) with a cosmetically or pharmaceutically acceptable organic phase; and C) subjecting the mixture of step B) to shear sufficient to induce limited coalescence. The Pickering emulsion prepared by the foregoing process is also contemplated in the present technology.

In an embodiment, step A) in the process of preparing the Pickering emulsion having a uniform size distribution can include, for example, (i) dissolving the polymer of the two-part stabilizer system into the continuous phase to prepare a polymer solution and (ii) homogenizing the inorganic solid particle of the two-part stabilizer system into the polymer solution.

Step C) in the above process of preparing the Pickering emulsion having a uniform size distribution can involve subjecting the mixture of step B) to shear sufficient to induce limited coalescence. Shear sufficient to induce limited coalescence means shear sufficient to generate particle droplets 3 to 10 times smaller than the ultimate size desired. In an embodiment, sufficient shear to induce limited coalescence can be achieved, for example, with a high shear mixer. In another embodiment, sufficient shear to induce limited coalescence can be achieved with a colloid mill. In a further embodiment, sufficient shear to induce limited coalescence can be achieved with a microfluidizer, a homogenizer, or ultrasonic energy.

In one embodiment, the mixture of B) is mixed in a high shear mixer, sufficient to induce limited coalescence. In some embodiments, step C) in the process of preparing the Pickering emulsion having a uniform size distribution can include (i) homogenizing the mixture of step B) to prepare an emulsion, followed by (ii) microfluidizing the emulsion.

In the “polymer suspension” technique, a suitable polymer is dissolved in a solvent and this solution is dispersed as fine water-immiscible liquid droplets in an aqueous solution that contains the inorganic solid, such as colloidal silica, as a stabilizer. Equilibrium is reached and the size of the droplets is stabilized by the action of the colloidal silica coating the surface of the droplets. The solvent is removed from the droplets by evaporation or other suitable technique resulting in polymeric particles having a uniform coating thereon of the inorganic solid particle. This process is further described in U.S. Pat. No. 4,833,060 issued May 23, 1989, incorporated by reference.

In another embodiment, step A) of the process of producing the Pickering emulsion can include an intermediate step of dissolving the polymer of the stabilizer system in a solvent, followed by dissolving or dispersing the dissolved polymer into an aqueous phase along with the solid particles. After a period of time sufficient to achieve limited coalescence equilibrium, the aqueous phase of step A) can be mixed in step B) with the cosmetically or pharmaceutically acceptable organic phase.

### Cosmetically or Pharmaceutical Compositions

Another aspect of the technology includes a cosmetically or pharmaceutically acceptable skin-care composition. The skin-care composition can include A) an aqueous phase, or a cosmetically or pharmaceutically acceptable organic phase; and B) a Pickering emulsion as set forth above.

The cosmetically or pharmaceutically acceptable skin-care composition can additional include at least one cosmetically or pharmaceutically acceptable additive.

It is known that formulated compositions for personal care and topical, dermatological, health care, which are applied to the skin and mucous membranes for cleansing or soothing, are compounded with many of the same or similar physiologically tolerable ingredients and formulated in the same or similar product forms, differing primarily in the purity grade of ingredients selected, by the presence of medicaments or pharmaceutically accepted compounds, and by the controlled conditions under which products may be manufactured. It is also known that the selection and permitted amount of ingredients also may subject to governmental regulations, on a national, regional, local, and international level.

The choice and amount of ingredients in formulated compositions containing the uniform dispersion of a stabilizer system will vary depending on the product and its function, as is well known to those skilled in the art. Formulation ingredients for personal care and topical health care products can typically include, but are not limited to, solvents, surfactants (as cleansing agents, emulsifying agents, foam boosters, hydrotropes, solubilizing agents, and suspending agents), non-surfactant suspending agents, emulsifiers, skin conditioning agents (emollients, moisturizers, and the like), film-formers, skin protectants, binders, chelating agents, antimicrobial agents, antifungal agents, abrasives, adhesives, absorbents, colorants, deodorants agents, antiperspirant agents, humectants, opacifying and pearlescing agents, antioxidants, preservatives, propellants, spreading agents, sunscreen agents, sunless skin tanning accelerators, ultraviolet light absorbers, pH adjusting agents, botanicals, hair colorants, oxidizing agents, reducing agents, skin bleaching agents, pigments, physiologically active agents, anti-inflammatory agents, topical anesthetics, fragrance and fragrance solubilizers, and the like, in addition to ingredients previously described that may not appear herein. An extensive listing of substances and their conventional functions and product categories appears in the CFTA Dictionary, generally, and in Vol 2, section 4 and 5, in particular.

In an embodiment, the cosmetically or pharmaceutically acceptable skincare composition can be a sunscreen. In some embodiments, the cosmetically or pharmaceutically acceptable skin-care composition can reduce the transmission of UV light to a substrate coated with the composition.

A skin-care composition including the Pickering emulsion disclosed herein can provide improved moisturization, softness, lubricity, sensory properties (i.e., skin feel), water repellency, gloss, and surface properties, to name a few.

A skin-care composition including the Pickering emulsion disclosed herein can also provide improved free-radical protection. Free radical protection refers to the ability of the described invention to protect the encapsulated phase from degradation by free radicals. In skincare, and suncare in particular, the internal (oil) phase of the emulsions often contains compounds such as actives or UV filters that are susceptible to degradation by hydroxyl, peroxyl, or other free radicals. By creating a physical barrier around the emulsion, the described technology can minimize the diffusion of these radical species into the internal phase, thus increasing the stability of the encapsulated actives.

Unless otherwise indicated, each chemical or composition referred to herein should be interpreted as being a commercial grade material which may contain the isomers, by-products, derivatives, and other such materials which are normally understood to be present in the commercial grade.

It is known that some of the materials described above may interact in the final formulation, so that the components of the final formulation may be different from those that are initially added. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

The technology herein is useful for preparing near monodisperse and tunable emulsion droplets that can be uniformly coated onto both hydrophilic surfaces and hydrophobic surfaces, which may be better understood with reference to the following examples.

### EXAMPLES

### Example 1

Formulation

Water and MAE were added to a beaker and mixed with a Heidolph mixer and marine blade at 500 rpm until the MAE was dissolved. Silica was added to the beaker and the mixture was mixed using an IKA Ultra-Turrax T-25 homogenizer at 9,400 rpm for 40 seconds. After the 40 second mix, the mix was kept at 9,400 rpm while oil was added slowly. When all oil was added, mixing was increased to 13,400 rpm for 4 minutes. After the 4 minute period, the mix was kept at 13,400 rpm and preservative was added and mixing maintained for an additional 1 minute. The final emulsion was then run through a Divtech M-110P microfluidizer at 10,000 psi for 1 pass.

As can be seen in the table below, by adjusting the amount of MAE polymer and silica in the emulsion, the particle size can be tuned controllably.

| | Particle | | | | | |
|---|---|---|---|---|---|---|
| | Size | Water | MAE | Silica | Oil | Preservative |
| Sample | (μm) | (wt %) | (wt %) | (wt %) | (wt %) | (wt %) |
| 1 | ~10 | 67.14 | 2.06 | 0.5 | 30 | 0.3 |
| 2 | ~5 | 64.59 | 4.11 | 1 | 30 | 0.3 |
| 3 | ~2.5 | 59.48 | 8.22 | 2 | 30 | 0.3 |

### Example 2

Formulation—Water and MAE were added to a beaker and mixed with a Heidolph mixer and marine blade at 500 rpm until the MAE was dissolved. Separately, ingredients in Part A (below), were mixed and heated until a temperature of 65 C was reached. Silica was added to the beaker and the mixture was mixed using an IKA Ultra-Turrax T-25 homogenizer at 9,400 rpm for 40 seconds, and then heated to 65 C. After the 40 second mix, the mix was kept at 9,400 rpm while the oil phase, Part A, was added slowly. When all oil was added, the mixture was cooled to room temperature. The mixing speed was then increased to 13,400 rpm for 5 minutes. After 4 minutes, the mixture was kept at 13,400 rpm and Part B (below) was added and mixing maintained for an additional 1 minute. The final emulsion was then run through a Divtech M-110P microfluidizer at 10,000 psi for 1 pass. Part C (below) was then added, and mixed until uniform.

Table of Ingredients

| | | Sample | Sample | Sample | Control |
|---|---|---|---|---|---|
| | Ingredient | 4 | 5 | 6 | 1 |
| Aqueous | DI water (wt %) | 67.29 | 64.74 | 59.63 | 68.85 |
| phase | MAE (wt %) | 2.06 | 4.11 | 8.22 | — |
| | Silica (wt %) | 0.50 | 1.00 | 2.00 | — |
| Part A | PEG-20 Methyl Glucose Sesquistearate | — | — | — | 0.75 |
| | (wt %) | | | | |
| | Methyl Glucose Sesquistearate (wt %) | — | — | — | 0.25 |
| | Neopentyl Glycol Diethylhexanoate | 5.00 | 5.00 | 5.00 | 5.00 |
| | (wt %) | | | | |
| | Diisostearyl Malate (wt %) | 0.75 | 0.75 | 0.75 | 0.75 |
| | Triisostearoyl Polyglyceryl-3 Dimer | 0.50 | 0.50 | 0.50 | 0.50 |
| | Dilinoleate (wt %) | | | | |
| | Benzophenone-3 (Oxybenzone) (wt %) | 5.00 | 5.00 | 5.00 | 5.00 |
| | Octocrylene (wt %) | 6.00 | 6.00 | 6.00 | 6.00 |
| | Butyl Methoxydibenzoylmethane (Avo- | 2.50 | 2.50 | 2.50 | 2.50 |
| | benzone) (wt %) | | | | |
| | Homosalate (wt %) | 5.00 | 5.00 | 5.00 | 5.00 |
| | Octinoxate (Octyl Methoxycinnamate) | 3.00 | 3.00 | 3.00 | 3.00 |
| | (wt %) | | | | |
| | DL-α-tocopherol acetate (wt %) | 0.50 | 0.50 | 0.50 | 0.50 |
| Part B | DMDM Hydantoin (wt %) | 0.35 | 0.35 | 0.35 | 0.35 |
| Part C | Disodium EDTA (wt %) | 0.05 | 0.05 | 0.05 | 0.05 |
| | Ascorbic Acid (wt %) | 0.50 | 0.50 | 0.50 | 0.50 |
| | Polyvinyl Alcohol (wt %) | 1.00 | 1.00 | 1.00 | 0.0 |

Ultraviolet (UV) transmittance data of dried sunscreen films was measured in order to determine the Sun Protection Factor (SPF) of a sunscreen. The lower the transmittance that is measured, the more UV light is being blocked by the sunscreen and the higher the SPF value will be. As can be seen in the table below, all of the example sunscreens had lower transmittance than a conventional sunscreen made with the same amount of UV filters in the emulsion.

| Wave- | Transmit- | Transmit- | Transmit- | Transmit- |
|---|---|---|---|---|
| length | tance % | tance % | tance % | tance % |
| nm | Sample 4 | Sample 5 | Sample 6 | Control 1 |
| 290 | 11.43479 | 4.65682 | 5.26177 | 54.87016 |
| 300 | 11.36797 | 4.72087 | 5.28562 | 54.46175 |
| 310 | 11.49394 | 4.8473 | 5.40931 | 54.71822 |
| 320 | 11.9435 | 5.16348 | 5.74293 | 55.19642 |
| 330 | 13.33799 | 6.24472 | 6.85265 | 56.63083 |
| 340 | 15.11222 | 7.77013 | 8.34348 | 58.48642 |
| 350 | 16.75905 | 9.24845 | 9.73323 | 60.3677 |
| 360 | 18.71387 | 11.07952 | 11.39231 | 62.65134 |
| 370 | 22.27108 | 14.70275 | 14.59766 | 66.18587 |
| 380 | 26.75122 | 19.61469 | 18.90904 | 70.00406 |
| 390 | 43.62736 | 39.03817 | 36.64196 | 79.71048 |
| 400 | 84.0079 | 82.1312 | 79.88199 | 92.91526 |
| 410 | 95.47239 | 93.8451 | 92.26193 | 95.31617 |
| 420 | 97.05831 | 95.6099 | 94.27765 | 95.53828 |
| 430 | 97.46646 | 96.17757 | 95.03868 | 95.55071 |
| 440 | 97.83759 | 96.66371 | 95.68333 | 95.69033 |
| 450 | 98.52627 | 97.14879 | 96.7162 | 96.16698 |

### Example 3

Preparation of Dipropylene Glycol Adipate (DGA): To a 4-neck round bottom flask equipped with a mechanical stirrer, thermocouple, nitrogen inlet and condenser was charged 77 grams adipic acid, 53 grams dipropylene glycol, 0.4 grams methanesulfonic acid (70%) and 0.3 grams hypophosphorous acid (50%). The mixture was agitated at 300 rpm, heated to 160° C. and held with nitrogen sparge at 250 mL/min. After 6.5 hours, acid number was titrated as 125.6. The mixture was cooled to 80° C. and filtered through 75 micron filter bag to give a viscous liquid which turned milky and precipitated on standing at room temperature.

Water and DGA were added to a beaker and mixed with a Heidolph mixer and marine blade at 500 rpm until the DGA was well dispersed. Silica was added to the beaker and the mixture was mixed using an IKA Ultra-Turrax T-25 homogenizer at 9,400 rpm for 40 seconds. After the 40 second mix, the mix was kept at 9,400 rpm while sunflower oil was added slowly. When all oil was added, mixing was increased to 13,400 rpm for 4 minutes. After the 4 minute period, the mix was kept at 13,400 rpm and preservative was added and mixing maintained for an additional 1 minute. The final emulsion was then run through a Divtech M-110P microfluidizer at 10,000 psi for 1 pass. The resulting emulsion had a narrow size distribution, as described below:

| | Particle | | | | | |
|---|---|---|---|---|---|---|
| | Size | Water | DGA | Silica | Oil | Preservative |
| Sample | (μm) | (wt %) | (wt %) | (wt %) | (wt %) | (wt %) |
| 7 | 18 | 68.20 | 1.00 | 0.5 | 30 | 0.3 |

### Example 4

Preparation of dipropylene glycol glutarate (DPG): To a 4-neck, 1-liter round bottom flask equipped with a mechanical stirrer, thermocouple, nitrogen inlet and condenser was charged 324 grams Glutaric acid and 276 grams acetic anhydride. The mixture was agitated at 300 rpm, heated to 130° C. and held for 2 hours to distill acetic acid. When the distillation stopped, the mixture was cooled to 90° C., treated with 244 grams dipropylene glycol, heated back to 145° C. and held for another 3 hours. The acid number was titrated as 236.4. The mixture was cooled to 80° C. and filtered through 75 micron filter bag to give a viscous and clear liquid.

Water and DPG were added to a beaker and mixed with a Heidolph mixer and marine blade at 500 rpm until the DPG was well dispersed. Silica was added to the beaker and the mixture was mixed using an IKA Ultra-Turrax T-25 homogenizer at 9,400 rpm for 40 seconds. After the 40 second mix, the mix was kept at 9,400 rpm while sunflower oil was added slowly. When all oil was added, mixing was increased to 13,400 rpm for 4 minutes. After the 4 minute period, the mix was kept at 13,400 rpm and preservative was added and mixing maintained for an additional 1 minute. The final emulsion was then run through a Divtech M-110P microfluidizer at 10,000 psi for 1 pass. The resulting emulsion had a narrow size distribution, as described below:

| | Particle | | | | | |
|---|---|---|---|---|---|---|
| | Size | Water | DPG | Silica | Oil | Preservative |
| Sample | (μm) | (wt %) | (wt %) | (wt %) | (wt %) | (wt %) |
| 8 | 15 | 68.2 | 1 | 0.5 | 30 | 0.3 |

### Example 5

The stability of emulsions prepared according to the present technology was tested according to a test procedure adapted from the literature [1]. In brief, a liquid phase was prepared by creating a 1 mg/mL stock solution of a lipid peroxidation sensor (BODIPY® 665/676 dye purchased from Life Technologies) in dichloromethane. 90 μL of this solution were added to 30 g of hydrogenated polydecene (Puresyn™ 4) to create an oil phase. This oil phase was then protected from light, and used in both the control and experimental emulsions. Zhao, Y., Engineering of Barrier Properties of Colloidosome Interface to Reduce Oxidation and Control the Release of Encapsulants, in Department of Food Science. 2013, Drexel University: Philadelphia.

Formula 1 (comparative) was created using the ingredients described in Table 1 below. Methyl Glucose Sesquistearate and PEG-20 Methyl Glucose Sesquistearate were added to the oil phase, and heated to 60-65 C. This was then combined with deionized water heated to 60-65 C, and mixed using an IKA Ultra-Turrax T-25 homogenizer at 9,400 rpm for 5 minutes. The emulsion was then allowed to cool to room temperature.

Formula 2 was created using the ingredients described in Table 1 below. Water and dipropylene glycol glutarate (DPG) were added to a beaker and mixed with a Heidolph mixer and marine blade at 500 rpm until the DPG was dissolved. Silica was added to the beaker and the mixture was mixed using an IKA Ultra-Turrax T-25 homogenizer at 9,400 rpm for 40 seconds. After the 40 second mix, the mix was kept at 9,400 rpm while oil was added slowly. When all oil was added, mixing was increased to 13,400 rpm for 4 minutes. After the 4 minute period, the mix was kept at 13,400 rpm and preservative was added and mixing maintained for an additional 1 minute. The final emulsion was then run through a Divtech M-110P microfluidizer at 10,000 psi for 1 pass. Polyvinylalcohol was added to the mixture and mixed with a Heidolph mixer and marine blade at 500 rpm for 5 minutes. The pH of the emulsion was adjusted to pH 6-7 using sodium hydroxide.

TABLE 1

| Ingredient | Formula 1 | Formula 2 |
|---|---|---|
| Oil phase (described above) (wt %) | 30.00 | 30 |
| Deionized Water (wt %) | 67 | 60 |
| Silica (wt %) | 0 | 1 |
| DPG (wt %) | 0 | 4 |
| Polyvinylalcohol (20 wt % solution) (wt %) | 0 | 5 |
| Methyl Glucose Sesquistearate (wt %) | 1.50 | 0 |
| PEG-20 Methyl Glucose Sesquistearate (wt %) | 1.50 | 0 |

To test the stability of the emulsions to radicals, 0.75 mL of emulsion was combined with 0.75 mL of a 80 mM solution of 2,2′-azobis(2-methylpropionamidine)dihydrochloride (AAPH), purchased from Sigma Aldrich, in a quartz cuvette. The cuvette was immediately placed in a Fluoromax 4 fluorometer (Horiba Scientific). The cuvette was excited at 630 nm, with emission measured at 699 nm. The data was normalized for each sample, and corrected to account for sample densification due to creaming. Normalized corrected fluorescence is shown in Table 2

Data table of degradation data showing the Pickering emulsion provides improvement over standard emulsions

TABLE 2

| Normalized | | |
|---|---|---|
| Corrected | | |
| Fluorescence | Formula 1 | Formula 2 |
| At 0 s | 1.00 | 1.00 |
| At 1800 s | 0.90 | 0.99 |
| At 3600 s | 0.67 | 0.96 |

### Example 6

Data was gathered to support the use of the emulsion with silica coated TiO₂. Water and silica-coated titanium dioxide (Si—TiO2) core-shell nanoparticle were added to a beaker and sonicated at 73 W for 15 minutes. DPG was added to the beaker and the mixture was mixed with a Heidolph mixer and marine blade at 500 rpm until the DPG was dissolved. The mixture was then mixed using an IKA Ultra-Turrax T-25 homogenizer at 9,400 rpm for 40 seconds. After the 40 second mix, the mix was kept at 9,400 rpm while sunflower oil was added slowly. When all oil was added, mixing was increased to 13,400 rpm for 4 minutes. After the 4 minute period, the mix was kept at 13,400 rpm and preservative was added and mixing maintained for an additional 1 minute. The final emulsion was then run through a Divtech M110P microfluidizer at 10,000 psi for 1 pass. The resulting emulsion had a narrow size distribution, as described below:

| | Particle | | | | | |
|---|---|---|---|---|---|---|
| | Size | Water | DPG | Si—TiO2 | Oil | Preservative |
| Sample | (μm) | (wt %) | (wt %) | (wt %) | (wt %) | (wt %) |
| 6-1 | 80 | 66.7 | 2 | 1 | 30 | 0.3 |
| 6-2 | 38 | 65.7 | 2 | 2 | 30 | 0.3 |
| 6-4 | 22 | 63.7 | 2 | 4 | 30 | 0.3 |

### Example 7

Data was gathered to support the use of the emulsion with silica coated zinc oxide. Water and silica-coated zinc oxide (Si—ZnO) core-shell nanoparticle were added to a beaker and sonicated at 73 W for 15 minutes. DPG was added to the beaker and the mixture was mixed with a Heidolph mixer and marine blade at 500 rpm until the DPG was dissolved. The mixture was then mixed using an IKA Ultra-Turrax T-25 homogenizer at 9,400 rpm for 40 seconds. After the 40 second mix, the mix was kept at 9,400 rpm while sunflower oil was added slowly. When all oil was added, mixing was increased to 13,400 rpm for 4 minutes. After the 4 minute period, the mix was kept at 13,400 rpm and preservative was added and mixing maintained for an additional 1 minute. The final emulsion was then run through a Divtech M-100P microfluidizer at 10,000 psi for 1 pass. The resulting emulsion had a narrow size distribution, as described below:

| | Particle | | | | | |
|---|---|---|---|---|---|---|
| | Size | Water | DPG | Si—ZnO | Oil | Preservative |
| Sample | (μm) | (wt %) | (wt %) | (wt %) | (wt %) | (wt %) |
| 7-1 | 128 | 66.7 | 2 | 1 | 30 | 0.3 |
| 7-2 | 96 | 65.7 | 2 | 2 | 30 | 0.3 |
| 7-4 | 42 | 63.7 | 2 | 4 | 30 | 0.3 |

Each of the documents referred to above is incorporated herein by reference, including any prior applications, whether or not specifically listed above, from which priority is claimed. The mention of any document is not an admission that such document qualifies as prior art or constitutes the general knowledge of the skilled person in any jurisdiction. Except in the Examples, or where otherwise explicitly indicated, all numerical quantities in this description specifying amounts of materials, reaction conditions, molecular weights, number of carbon atoms, and the like, are to be understood as modified by the word “about.” It is to be understood that the upper and lower amount, range, and ratio limits set forth herein may be independently combined. Similarly, the ranges and amounts for each element of the invention can be used together with ranges or amounts for any of the other elements.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the essential or basic and novel characteristics of the composition or method under consideration.

While certain representative embodiments and details have been shown for the purpose of illustrating the subject invention, it will be apparent to those skilled in this art that various changes and modifications can be made therein without departing from the scope of the subject invention. In this regard, the scope of the invention is to be limited only by the following claims.

1. A Pickering emulsion, said Pickering emulsion being a finely dispersed water-in-oil or oil-in-water system, said Pickering emulsion comprising:
A) a cosmetically or pharmaceutically acceptable organic phase;
B) an aqueous phase; and
C) a stabilizer system,
wherein the Pickering emulsion has a uniform size distribution.

2. The Pickering emulsion of claim 1, wherein the Pickering emulsion generates a uniform film as evidenced by the generation of a diffraction pattern exhibiting one or more distinct rings rather than diffuse scattering when coated as a thin film on a transparent surface and illuminated with a coherent beam of light.

3. The Pickering emulsion of claim 2, wherein the transparent surface is a hydrophobic surface.

4. The Pickering emulsion of claim 1, wherein the stabilizer system comprises a non-ionic polymer and inorganic solid particles.

5. The Pickering emulsion of claim 4, wherein the inorganic solid particles comprise zinc oxide, titanium dioxide, silica, or a combination thereof.

6. The Pickering emulsion of claim 4, wherein said non-ionic polymer is a reaction product of a diol and a diacid.

7. The Pickering emulsion of claim 1, wherein said Pickering emulsion is prepared by the steps comprising:
A) dissolving the stabilizer system into at least one of the aqueous phase or the cosmetically or pharmaceutically acceptable organic phase;
B) slowly adding into the solution from step A) the other of the aqueous phase or the cosmetically or pharmaceutically acceptable organic phase with mixing;
C) subjecting the mixture of step B) to high shear sufficient to induce limited coalescence.

8. A cosmetically or pharmaceutically acceptable skin-care composition comprising:
A) a continuous phase comprising water, or a cosmetically or pharmaceutically acceptable organic phase; and
B) the Pickering emulsion of claim 1.

9. The cosmetically or pharmaceutically acceptable skin-care composition of claim 8, further comprising at least one cosmetically or pharmaceutically acceptable additive.

10. The cosmetically or pharmaceutically acceptable skin-care composition of claim 8, wherein the at least one cosmetically or pharmaceutically acceptable additive comprises a UV absorber.

11. A process for producing a Pickering emulsion having a uniform size distribution comprising:
A) dissolving or dispersing a stabilizer system into an aqueous phase;
B) slowly mixing the aqueous phase of step A) with a cosmetically or pharmaceutically acceptable organic phase, and
C) subjecting the mixture of step B) to shear sufficient to induce limited coalescence.

12. The process of claim 11, wherein step A) comprises (i) dissolving a stabilizer into the continuous phase to prepare a stabilizer solution, and (ii) homogenizing a solid particle into the stabilizer solution.

13. The process of claim 11 or 12, wherein step C) comprises
(i) homogenizing the mixture of step B) to prepare an emulsion, followed by
(ii) microfluidizing the emulsion.
